(19) United States
(12) Patent Application Publication
(10) Pub. No.: US 2013/0053673 A1
(43) Pub. Date: Feb. 28, 2013

(54) CYSTIC FIBROSIS DIAGNOSTIC DEVICE AND METHOD

(71) Applicant: Philippe Brunswick, Paris (FR)

(72) Inventor: Philippe Brunswick, Paris (FR)

(73) Assignee: IMPETO MEDICAL, Paris (FR)

(21) Appl. No.: 13/215,788

(22) Filed: Aug. 23, 2011

(51) Int. Cl. A61B 5/053; A61B 5/04

(52) U.S. Cl. 600/384; 600/547

(57) ABSTRACT

A method for detecting cystic fibrosis is disclosed, which is performed in a system comprising an anode and a cathode placed on different regions of the patient body, and an adjustable DC source, which is controlled in order to feed the anode with a DC current. The method includes applying DC voltage pulses of varying voltage values to the anode for given durations allowing the stabilization of electrochemical phenomena in the body in the vicinity of the electrodes, collecting data representative of the current between the electrodes, and of the potentials of the electrodes, for the different DC voltages, and from the data, computing data representative of the electrochemical skin conductance of the patient, and reconciling the latter data with reference data obtained in the same conditions on patients suffering or not from cystic fibrosis, and identifying the patient as suffering or not from cystic fibrosis.

### BACKGROUND OF THE INVENTION

1. Field of the Invention

The invention relates in general to medical diagnostic devices and methods in the field of human health. The invention more specifically applies to diagnostic of cystic fibrosis.

2. Description of the Related Art

Cystic fibrosis is the most frequent lethal autosomal recessive disorder in Caucasians. It is characterized by defective electrolyte transport across secreting epithelia. It is due to mutations in the Cystic Fibrosis Conductance Transmembrane Regulator (CFTR) gene which encodes a chloride channel that also regulates other ion channels.

Cystic fibrosis diagnosis is based on the presence of one or more characteristic phenotypic features and laboratory evidence showing elevated sweat chloride concentration, as is explained in these publications:
Gibson L E, Cooke R E. A test for concentration of electrolytes in sweat in cystic fibrosis of the pancreas utilizing pilocarpine by iontophoresis. Pediatrics 1959;23:545-9.
Di Sant'Agnese P A, Darling R C, Perera G A, Shea E. Abnormal electrolyte composition of sweat in cystic fibrosis of the pancreas; clinical significance and relationship to the disease. Pediatrics 1953;12:549-63.

Yet there are several critical issues associated with sweat testing that can challenge the evaluation of a sweat test result. A reliable sweat test depends on the use of appropriate methods, as described in various guidelines, performed by documented competent laboratories with active quality control and quality assurance programs.

Some of these guidelines are:
Baumer J H. Evidence based guidelines for the performance of the sweat test for the investigation of cystic fibrosis in the UK. Arch Dis Child 2003;88:1126-7,
LeGrys V A. Sweat testing for the diagnosis of cystic fibrosis: practical considerations. J Pediatr 1996;129:892-7, and
LeGrys V A, Yankaskas J R, Quittell L M, Marshall B C, Mogayzel Jr P J. Diagnostic sweat testing: the Cystic Fibrosis Foundation guidelines. J Pediatr 2007;151:85-9.

These guidelines can be very plodding and time-consuming to follow.

### SUMMARY OF THE INVENTION

Thus, one object of the present invention is to provide a new technology with immediate assessment of CFTR-related chloride transport to evaluate cystic fibrosis or CFTR-related disease likelihood. Another object of the invention is to provide a non-invasive measurement that is quick and easy to implement on a patient.

According to the invention, a method for diagnosing a patient, with a view to detecting cystic fibrosis is provided. The method according to the invention is carried out in a system comprising an anode and a cathode, intended to be placed on different regions of the patient body, and an adjustable DC source, which is controlled in order to feed the anode with a DC current.

In a preferred embodiment, the method comprises the steps consisting of:
applying DC voltage pulses of varying voltage values to the anode for given durations allowing the stabilization of electrochemical phenomena in the body in the vicinity of the electrodes,
collecting data representative of the current between the anode and the cathode, and of the potentials of the anode and the cathode, for the different DC voltages,
from said data, computing data representative of the electrochemical skin conductance of the patient,
reconciling said data representative of the electrochemical skin conductance of the patient with reference data obtained in the same conditions on patients identified as suffering or not from cystic fibrosis, and identifying the patient as suffering or not from cystic fibrosis.

The electrochemical skin conductance value at a given voltage applied on the anode may be determined as the ratio between the current through the anode and the cathode and the voltage difference between the anode and the cathode. The computed data relative to the electrochemical skin conductance values of the patient may include the difference and/or the ratio between two electrochemical skin conductance values of the patient for two different voltage values applied to the anode.

In additional embodiments, the present invention includes a system for diagnosing a patient, with a view to detecting cystic fibrosis, comprising:
an anode and a cathode, intended to be placed on different regions of the patient body,
an adjustable DC source, which is controlled in order to feed the anode with pulses of a DC current of varying voltage values, for given durations allowing the stabilization of electrochemical phenomena in the body in the vicinity of the electrodes,
a measuring circuit, designed to collect data representative of the current between the anode and the cathode, and of the potentials of the anode and the cathode, for the different DC voltages,
wherein the system further comprise a computing circuit, designed to compute data representative of the electrochemical skin conductance of the patient and to reconcile said data with reference data obtained in the same conditions on patients identified as suffering or not from cystic fibrosis.

### BRIEF DESCRIPTION OF THE DRAWINGS

The features and advantages of the invention will be apparent from the following more detailed description of certain embodiments of the invention and as illustrated in the accompanying drawings, in which:

FIG. 1 shows a system for diagnosing a patient according to the invention.

FIG. 2 shows the main steps carried out in the diagnosis method provided by the invention.

FIG. 3 is an exemplary display of data representative of electrochemical skin conductance for a patient affected of cystic fibrosis and a control patient.

FIGS. 4a and 4b show comparative data representative of electrochemical skin conductance measured on hands and feet of control patients and patients affected of cystic fibrosis.

FIG. 5 shows relation between chloride concentrations and data representative of electrochemical skin conductance for control patients and patients affected of cystic fibrosis.

### DETAILED DESCRIPTION

The defect in the CFTR chloride channel results in an altered bioelectrical potential in the cystic fibrosis sweat gland. Thus sweat chloride movements upon electric stimulation, and therefore the electrochemical skin conductance, are impaired in patients suffering from cystic fibrosis. This is the basis of the invention, which explores the sweat duct using active electrophysiology, such as measurement of electrochemical skin conductance after application of a low direct voltage via nickel electrodes.

This low voltage applied on human skin generates a current through reverse iontophoresis, i.e. ion movements via sweat duct pores in response to electric stimulation and local electrochemical reactions between those ions and nickel electrodes. On human skin, at the voltages applied in the current invention (i.e. below 10 V), ions cannot go through the stratum corneum because of the very high electrical capacity of its dense lipid layers. Thus, the only way for ions in human skin to move in such an electric field is via sweat duct pores, and the measurement of electrochemical skin conductance as carried out in the present invention is correlated to the sweat chloride movements impaired by cystic fibrosis, and is as such a good tool for its diagnosis.

### Description of a Diagnosis System According to the Invention

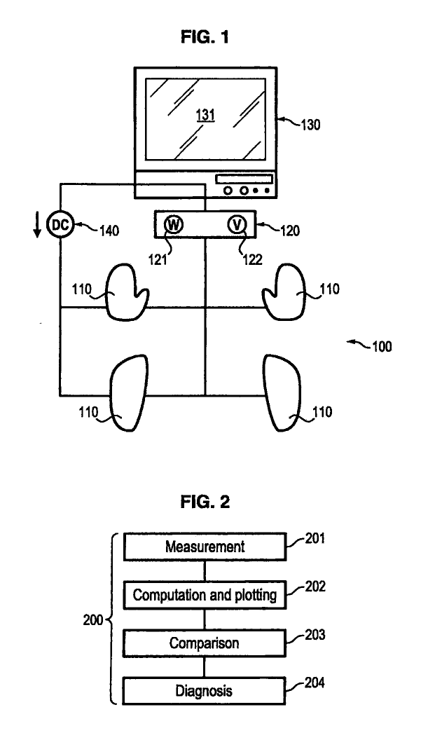

A system 100 for diagnosing a patient by assessing electrochemical skin conductance is shown in FIG. 1. The system 100 comprises a series of large area electrodes 110, preferably four electrodes 110, on which the patient can place his hands and feet. The sites of the electrodes 110 have been chosen because of their high density of sweat glands.

The electrodes 110 can be made of nickel. Their individual surface area is comprised between 50 cm² and 200 cm², so that they cover substantially all the surface of the hand palms and of the feet soles. Yet they can be adapted for children or even infants.

The electrodes are connected to an electronic board 120 managed by a computer 130 for collecting, computing, and storing data. They are also connected to an adjustable DC source 140, which is controlled by an operator or the electronic board to feed the electrodes 110 with a DC current of a determined voltage.

The electronic board 120 is also designed to measure the voltage potential of each electrode through a voltmeter 121, as well as the current between two electrodes through a Wheatstone bridge 122. The diagnosing system can also be equipped with a display 131, designed for displaying the measured data as well as the results of the computations carried out on said data. The diagnosis method 200 according to the invention will now be described with reference to FIG. 2.

### Measurement Step 201

In order to diagnose a patient, in view of detecting cystic fibrosis, the patient places his hands and feet on the large area nickel electrodes 110, and stands up without moving his hands and feet during the 2 minutes that lasts the measurement. The measurement 201 is carried out independently for the two feet and for the two hands. For one measurement configuration (for example right-hand, left-hand), one electrode is used as an anode, and the other one is used as a cathode.

The anode is then fed with DC current. The anode is applied an initial voltage, comprised between 0.5 V and 1.5 V, preferably equal or close to 1 V, during a duration comprised between 0.5 second and 2 seconds, preferably 1 second. The duration must last long enough to allow the stabilization of electrochemical phenomena in the body, in the vicinity of the electrodes.

The applied current induces voltage on the cathode, and a current going through the body towards the cathode, as previously explained. Both electrodes voltages and current through them are measured and stored by the electronic board 120 at measurement step 201. Then, the voltage applied on the anode is increased by a step comprised between 0.1 V and 0.3 V, preferably 0.2 V.

For instance, the voltage applied is increased from 1 V up to 1.2 V. This voltage value is applied on the anode during between 0.5 second and 2 seconds, preferably 1 second, and a new measurement is performed. Such a progressive step by step increase of between 0.1 V and 0.3 V, preferably 0.2 V, during preferably 1 second is applied until a maximal voltage below 10V, preferably of between 3.5 V and 4 V, and even more preferably of 3.8 V is reached.

This stepwise increase represents preferably a total of 15 measurements, when the minimum voltage value is 1 V, the maximum voltage value is 3.8 V, and the step is 0.2 V. The following results have been obtained with these experiments conditions.

The same series of measurements can also be carried out in reverse, by applying successive pulses of decreasing voltages. The same series of measurements can then be carried out with the electrodes being reversed (anode becoming cathode and vice-versa), and the same can be carried out on the feet.

### Computation and Plotting 202

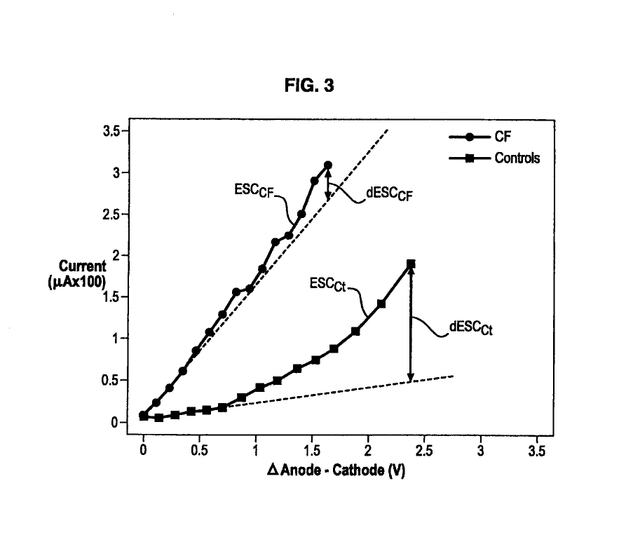

Once the electrodes potentials have been recorded, the electronic board computes the difference in voltages between the anode and the cathode, noted Δ(Anode-Cathode), for each DC voltage applied to the anode, as illustrated in FIG. 3. The current measured at each voltage is then plotted against the difference in voltages Δ(Anode-Cathode). The curve obtained is linear when voltage applied to the anode is low.

The electrochemical skin conductance, being the slope of the curve, i.e. the ratio between the current measured and the difference in voltages Δ(Anode-Cathode), is then computed. This step of computation and plotting is referenced as 202 on FIG. 2.

### Comparison with Healthy Patients 203

FIG. 3 shows the plot of current against the difference in voltages Δ(Anode-Cathode) for each voltage applied to the anode. The curve with squared plots represents measurements of a healthy patient, whereas the curve with round plots represents a patient suffering from cystic fibrosis.

As is clearly visible on FIG. 3, the electrochemical skin conductance in patients suffering from cystic fibrosis is higher than that of healthy patients when intermediate voltages are applied (between 0.5 V and 2 V). This phenomenon is most probably due to the high sweat chloride concentration in patients suffering from cystic fibrosis, which allow a larger concentration of electrochemical reaction with the nickel electrodes, as compared to healthy patients with low sweat chloride concentrations. In particular, amongst intermediate values, the 1.6 V value is the most discriminant one at low voltage, between a diseased patient and a healthy one. We define ESC as the electrochemical skin conductance when a voltage of 1.6 V is applied on the anode.

On FIG. 3, the 1.6 V value is the slope of the curve obtained at the fourth point of each curve. Thus the diagnosis can then be carried out by comparing the patient ESC with control or reference ESC.

In addition, at higher voltages, it is visible on FIG. 3 that, while there is an increase in electrochemical skin conductance in healthy patients, no such behaviour is observed in diseased patients. With higher voltages applied, the sweat gland physiology is likely to be overcome: although quite low (3.6 V), this voltage is much higher than the physiological one (in the mV range) and the ion channels in the sweat duct would consequently function towards ion efflux.

While a large efflux is possible in controls, it is limited in patients suffering from cystic fibrosis for whom sweat chloride is already at baseline in the maximal physiological range and for whom CFTR chloride channels in the sweat duct epithelium are poorly expressed/functional. This explains why at high voltages, there is no increase in electrochemical skin conductance in diseased patients, as compared with healthy subjects. To quantify this evolution, the difference dESC, or the ratio between electrochemical skin conductance calculated at a high voltage and an intermediate voltage is computed. Preferably, the difference dESC or the ratio is computed between the electrochemical skin conductance calculated at 3.6 V and ESC (obtained at 1.6 V).

The voltage step 3.6 V was chosen because it is one of the highest voltages applied and the most discriminant between diseased patients and healthy subjects. On FIG. 3, the 3.6 V value is the slope of the curve obtained on the 14^{th} point of each curve. dESC are shown on the figure by the up and down arrows.

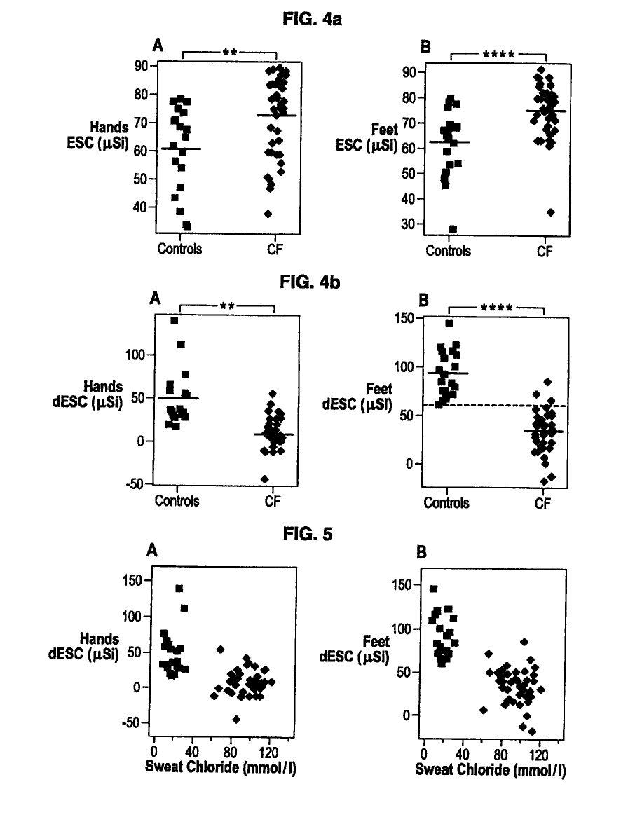

The diagnosis of cystic fibrosis can be carried out by comparing the feet dESC, or the ratio between electrochemical skin conductance obtained at 3.6 V and 1.6 V, between patients and healthy subjects, as it is the most discriminative measurement. Experiments carried out on study subjects (41 diseased patients, 20 healthy subjects) in the same conditions showed that feet dESC of a patient being below 60 μSi predicts cystic fibrosis, with a diagnostic specificity of 1 and a sensitivity of 0.93 as is more developed below in reference to FIG. 4b.

Alternatively, if the ratio between the electrochemical skin conductance values obtained at 3.6 V and 1.6 V, respectively, is determined to be greater than 2, this determines with high confidence that the patient is not affected by cystic fibrosis. As the most discriminant measurements are those carried out at 1.6 V and 3.6 V, the measurement step 202 carried out on the patient can be limited to the measurement of the anode and cathode potential, as well as the current flowing in between, during the application to the anode of two waves of current during 1 s each, and which voltages are respectively of 1.6 V and 3.6 V. The electronic board thus only computes the Δ(Anode-Cathode) at 1.6 V and 3.6 V, the electrochemical skin conductance of the patient at these voltages, and its dESC (difference between the electrochemical skin conductance at 3.6 V and 1.6 V).

### Statistical Analysis

More results of the broad range experiments are displayed on FIGS. 4a, 4b and 5. The following results are expressed by a mean ±SD. Means of each group were compared by a two sided Student's t-test. A p-value <0.05 was regarded as statistically significant. The repeatability was studied by the Bland-Altman method and the results expressed as mean [95% confidence interval]. The precision of the method, i.e. the mean of absolute values of difference between the two measurements performed for a subject divided by the average of the means of all measurements, was calculated. The diagnostic performance of electrochemical skin conductance measurement was analyzed by a receiver-operator characteristic (ROC) curve, allowing determining the sensitivity, specificity and the accuracy of the diagnostic test by the area under the ROC curve (AUC). A value of 0.5 under the ROC curve indicates that the variable performs no better than chance, a value >0.7 is considered “fair”, >0.8 as “good”, >0.9 as “excellent” and 1.0 indicates perfect discrimination.

In reference to FIG. 4a, the ESC (electrochemical skin conductance measurement when a voltage of 1.6 V is applied) is represented for hands (A) and feet (B) in control subjects and patients with cystic fibrosis. The line for each group is the mean value.

This figure shows that ESC on the hands is significantly different in patients with cystic fibrosis (73±14 μSi), as compared with control subjects (61±15 μSi, P<0.01). ESC measurements on the feet were also significantly different in patients with cystic fibrosis (75±10 μSi), as compared with control subjects (62±13 μSi, p<0.0001). However, for both hands and feet ESC, there was some overlap between the two groups.

In reference to FIG. 4b is represented dESC (difference in electrochemical skin conductance at 3.6 V and 1.6 V) on hands (A) and feet (B) in control subjects and patients with cystic fibrosis. The dotted line represents the cut-off of 60 μSi, the line for each group is the mean value. Although there was a broad range of results in hands and feet dESC measurements in both patients suffering from cystic fibrosis and control subjects, there was little overlap between the two groups.

dESC measurements on the hands were significantly different in patients with cystic fibrosis (9±18 μSi), as compared with control subjects (49±31 μSi, p<0.0001). dESC measurements on the feet were also significantly different in patients suffering from cystic fibrosis (34±24 μSi), as compared with control subjects (93±24 μSi, p<0.0001).

For feet dESC measurement and with a cut-off value <60 μSi predicting cystic fibrosis, all control subjects had a normal result and only three patients with cystic fibrosis had feet dESC above the cut-off value (65, 85 and 72 μSi). However, their hands dESC were all in the patient with cystic fibrosis range (−12, 9 and 0 μSi, respectively).

To study repeatability, a second set of measurements was performed and analyzed by the Bland-Altman method: the mean differences [95% CI] between the 2 measurements were: for hands ESC: 1.78 [0.44; 3.10] μSi, for feet ESC: −1.93 [−2.73; −1.14] μSi, for hands dESC: 3.30 [−4.10; 10.70] μSi, and for feet dESC: 1.72 [−3.25; 6.70] μSi. Thus, the precision of the measurements was very good: 0.06 for hands ESC, 0.04 for feet ESC, 0.09 for hands dESC and 0.03 for feet dESC.

As feet dESC was the most discriminative measurement, its diagnostic performance was analyzed by ROC curve modelisation. With a cut-off value <60 μSi predicting cystic fibrosis, dESC measurement provided a diagnostic specificity of 1, a sensitivity of 0.93 and a diagnostic performance as assessed by the area under a ROC curve of 0.96 [95% CI 0.92; 1.00].

dESC and sweat chloride concentrations were plotted in relation to each other, in reference to FIG. 5. Square plots are represent measurements on healthy subjects, and diamond plots represent measurement on patients with cystic fibrosis. dESC and sweat chloride concentrations were plotted in relation to each other. There was a good correlation of dESC to sweat chloride, both for hands: the coefficient of correlation was −0.57 (pb 0.0001) and for feet: coefficient of correlation: −0.70 (pb 0.0001).

### Diagnosis Step 204

In a nutshell, the results of the computation of ESC and/or dESC, preferably feet dESC of a patient can be used as a diagnostic tool, by comparison to results of healthy patients during a diagnostic step 204. The test is completely automatic and results are immediately displayed. It requires no special skill or training for its operator. It could be performed by the practitioner during an outpatient consultation, just as blood pressure measurement or pulmonary function tests.

It needs no patient preparation, it is painless and has no contra-indication. It only requires that the patient stands quietly during the 2 minutes measurement with hands and feet placed on the electrodes. If one takes into account the time for explanations, setting up the patient and registration of patient demography, the whole exam is performed in ten minutes.

It is noteworthy the maximal voltage applied is quite low (in the range of a usual battery) and the intensity of the current produced (0.2 mA) is much lower than the current intensity usually used when iontophoresing the potent sweat secretagogue pilocarpine on an electrically stimulated area of the skin (around 2 mA). The electrodes and the duration of measurement could be adapted for children or even infants.

What is claimed is:

1. A method for diagnosing a patient, with a view to detecting cystic fibrosis, the method being performed in a system comprising:
an anode and a cathode, intended to be placed on different regions of the patient body;
an adjustable DC source, which is controlled in order to feed the anode with a DC current;
the method comprising the following steps:
applying DC voltage pulses of varying voltage values to the anode for given durations allowing the stabilization of electrochemical phenomena in the body in the vicinity of the electrodes;
collecting data representative of the current between the anode and the cathode, and of the potentials of the anode and the cathode, for the different DC voltages;
from the data, computing data representative of the electrochemical skin conductance of the patient; and
reconciling the data representative of the electrochemical skin conductance of the patient with reference data obtained in the same conditions on patients identified as suffering or not from cystic fibrosis, and identifying the patient as suffering or not from cystic fibrosis.

2. A method according to claim 1, wherein the electrochemical skin conductance value at a given voltage applied on the anode is determined as the ratio between the current through the anode and the cathode and the voltage difference between the anode and the cathode.

3. A method according to claim 1, wherein the duration is comprised between 0.5 and 2 seconds, and preferably close to 1 second.

4. A method according to claim 1, wherein the voltage values of the pulses increase and/or decrease stepwise.

5. A method according to claim 4, wherein the step increase or decrease between two successive pulses is comprised between 0.1 and 0.3 V, and preferably close to 0.2 V.

6. A method according to claim 5, wherein the voltage values are in the range from about 0.5 V and 10 V, preferably between 1 V and 3.8 V.

7. A method according to claim 1, wherein the computed data relative to the electrochemical skin conductance values of the patient include the electrochemical skin conductance value of the patient as a function of the voltage value applied to the anode.

8. A method according to claim 1, wherein the computed data relative to the electrochemical skin conductance values of the patient include the electrochemical skin conductance value of the patient at a voltage value comprised between about 1.4 and 1.8 V, preferably close to 1.6 V.

9. A method according to claim 8, wherein the computed data relative to electrochemical skin conductance values of the patient also includes the electrochemical skin conductance value of the patient at a voltage comprised between about 3.4 V and 3.8 V.

10. A method according to claim 7, wherein the computed data relative to the electrochemical skin conductance values of the patient include the difference and/or the ratio between two electrochemical skin conductance values of the patient for two different voltage values applied to the anode.

11. A method according to claim 9, wherein the computed data relative to the electrochemical skin conductance values of the patient include the difference and/or the ratio between two electrochemical skin conductance values of the patient for an intermediate and a high voltage value applied to the anode.

12. A method according to claim 10, wherein the reconciling step comprises determining whether the difference and/or the ratio between two electrochemical skin conductance values of the patient for intermediate and high voltage values applied to the anode is below a given threshold.

13. A method according to claim 12, wherein the intermediate voltage is comprised between 1.4 V and 1.8 V, and is preferably close to 1.6 V, and the high voltage value is comprised between 3.4 V and 3.8 V, and is preferably close to 3.6 V.

14. A method according to claim 1, wherein the electrodes include hands and feet electrodes.

15. A method according to claim 14, wherein the electrodes cover substantially all the surface of the hand palms and of the feet soles.

16. A system for diagnosing a patient, with a view to detecting cystic fibrosis, comprising
an anode and a cathode, intended to be placed on different regions of the patient body;
an adjustable DC source, which is controlled in order to feed the anode with pulses of a DC current of varying voltage values, for given durations allowing the stabilization of electrochemical phenomena in the body in the vicinity of the electrodes;
a measuring circuit, designed to collect data representative of the current between the anode and the cathode, and of the potentials of the anode and the cathode, for the different DC voltages; and
wherein the system further comprises a computing circuit, designed to compute data representative of the electrochemical skin conductance of the patient and to reconcile the data with reference data obtained in the same conditions on patients identified as suffering or not from cystic fibrosis.

17. The system according to claim 16, wherein the voltage values are in the range from about 0.5 V and 4 V, preferably between 1 V and 3.8 V.

18. The system according to claim 17, wherein the DC source is designed to feed the anode with successive pulses of DC current which voltage increase stepwise.

19. The system according to claim 17, wherein the computing circuit calculates the ratio between the current through the anode and the cathode, and the difference in voltages between the anode and the cathode, as a function of the voltage applied to the anode, and wherein the system further includes a display that is designed for displaying said data as a curve.

20. The system according to claim 16, wherein the computing circuit is designed to compute the difference between two electrochemical skin conductance values of the patient for two different voltage values applied to the anode.

21. The system according to claim 20, wherein the computing circuit compares the difference between two electrochemical skin conductance values of the patient for intermediate and high voltage values applied to the anode to a given threshold.
